(19) United States
(12) Patent Application Publication
(10) Pub. No.: US 2024/0325642 A1
(43) Pub. Date: Oct. 3, 2024

(54) A subassembly of a medicament delivery device

(71) Applicant: SHL Medical AG, Zug (CH)

(72) Inventors: Ming-Ting Yin, Taoyuan City (TW); Philipp Zingel, Taoyuan City (TW); Roman Käslin, Zurich (CH); Lino Manuel Heckhorn Ghilardi, Zurich (CH)

(21) Appl. No.: 18/293,959

(22) PCT Filed: Aug. 3, 2022
(86) PCT No.: PCT/EP2022/071831

§ 371 Date: Jan. 31, 2024

(30) Foreign Application Priority Data
Aug. 9, 2021 (EP) 21190458.6

(51) Int. Cl. A61M 5/20; A61M 5/31; A61M 5/315

(52) U.S. Cl. CPC A61M 5/2033; A61M 5/31578; A61M 2005/3131; A61M 2005/3142; A61M 2205/19; A61M 2207/00

(57) ABSTRACT

A subassembly (100) of a medicament delivery device (102), the subassembly (100) comprising a housing (104) configured to contain a medicament container structure (250) from which medicament is expelled during use of the medicament delivery device (102), the housing (104) having a proximal end (106) and a distal end (108), and a drive assembly (110) comprising a plunger rod (124) at a proximal end (120) of the drive assembly (110), the plunger rod (124) being configured to, upon activation, apply an activating force on the medicament container structure (250) for expelling the medicament from the medicament delivery device (102), wherein the proximal end (106) of the housing (104) comprises an opening (112), and the distal end (108) comprises a sealable portion (114), wherein the drive assembly (110) is insertable into the housing (104) at the proximal end (106) to form the subassembly (100).

### CROSS-REFERENCE TO RELATED APPLICATIONS

The present application is a U.S. National Phase Application pursuant to 35 U.S.C. § 371 of International Application No. PCT/EP2022/071831 filed Aug. 3, 2022, which claims priority to EP patent application Ser. No. 21/190,458.6 filed Aug. 9, 2021. The entire disclosure contents of these applications are herewith incorporated by reference into the present application.

### TECHNICAL FIELD

The present disclosure generally relates to a subassembly of a medicament delivery device for expelling medicament from a medicament container structure.

### BACKGROUND

Medicament is conventionally expelled from a medicament delivery device to a so-called dose delivery site, e.g. through the skin of a person in need of the medicament. The medicament delivery device thus comprises a medicament container that contains the medicament. The medicament container is preferably arranged within a housing and is expelled when exposed to activation from e.g. a drive assembly.

These days, a number of different medicament delivery devices exist, including various types of pen injectors, autoinjectors and on-body devices. For example, a needle can be directly arranged on the medicament container, whereby a so-called rigid needle shield is arranged on the needle to protect the needle from exposure. The medicament container with the needle is, together with an activation mechanism, arranged in a housing configured for this type of configuration.

Although many of these devices have enabled major improvements in the management of a number of medical conditions, various limitations do still exist in the current technology. Not least amongst these are the robustness and mechanical properties of the medicament delivery device. Also, assembling of the medicament delivery device prior to usage thereof is an issue of further improvement. In considering these problems, the applicant has appreciated that various developments could be made to help improve the medicament delivery devices on the market today, which are set out in more detail below.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to a first aspect of the present disclosure there is provided a subassembly of a medicament delivery device, the subassembly comprising a housing configured to contain a medicament container structure from which medicament is expelled during use of the medicament delivery device, the housing having a proximal end and a distal end, and a drive assembly comprising a plunger rod at a proximal end of the drive assembly, the plunger rod being configured to, upon activation, apply an activating force on the medicament container structure for expelling the medicament from the medicament delivery device, wherein the proximal end of the housing comprises an opening, and the distal end comprises a sealable portion, wherein the drive assembly is insertable into the housing at the proximal end to form the subassembly.

The drive assembly should be construed as an arrangement which is configured to provide an activating force on the medicament container structure during use of the medicament delivery device. By means of the activating force, the medicament is expelled through a needle and into the dose delivery site. The activating force may either be generated by a manual actuation, or automatically when pushing the medicament delivery device to the dose delivery site.

The present disclosure is based on the insight that the mechanical strength of the housing can be increased by providing a sealable portion at the distal end of the housing. An advantage is thus that a stronger packaging shell is provided for the medicament delivery device. In particular, the rear end of the housing has an increased stiffness. Also, and as will be described in further detail below, the assembly process of the components forming part of the subassembly will be more efficient since all components are inserted from one and the same side of the housing, i.e. the proximal end of the housing. Since the housing is sealed at the distal end, there will also be no need of sealing the housing as a final step of an assembly process.

According to an exemplified embodiment, the housing may comprise an envelope surface extending axially from the distal end to the proximal end, the sealable portion being integrally formed with the envelope surface. Hereby, the sealable portion and the envelope surface are formed in one piece.

According to an exemplified embodiment, the sealable portion may be formed by a removable cap. The removable cap is thus connected to the distal end of the housing prior to inserting the drive assembly into the housing. The removable cap can be connected to the distal end of the housing at the production site producing the housing, or at the assembly site where the subassembly is assembled.

According to an exemplified embodiment, a distal end of the drive assembly may be arranged at the distal end of the housing when the drive assembly is inserted into the housing. The medicament container structure, when arranged in the housing, is thus arranged proximally from the drive assembly.

According to an exemplified embodiment, the housing may be a tubular-shaped housing, e.g. cylindrical housing or rectangular housing that are forming a hollow housing structure. Also, and according to an exemplified embodiment, the housing may comprise an inspection portion.

The inspection portion advantageously enables the user of the medicament delivery device to visually inspect the medicament currently present in the medicament container structure. The medicament container structure is thus preferably formed by a transparent material or comprising a counter inspection aperture aligned with the inspection portion of the housing. To be able to visually inspect the medicament container structure, the inspection portion may preferably be an elongated inspection portion extending along an axial direction of the housing, wherein a proximal end of the inspection portion is arranged closer to the proximal end of the housing compared to the distal end of the housing. Preferably, the elongated inspection portion has an extension that enables the user of the medicament delivery device to visually inspect the entire medicament container structure. Hence, the inspection portion preferably has a length in the axial direction, which length is longer than the axial extension of the medicament container structure.

According to an exemplified embodiment, the sealable portion may comprise a connector portion at a surface facing the drive assembly. Hereby, the drive assembly can be attached to the connector portion, thereby preventing the drive assembly from falling out of the housing before final assembly is completed.

According to an exemplified embodiment, the housing may comprise a drive assembly guiding structure, the drive assembly guiding structure extending in an axial direction on an inner surface of the housing. The drive assembly guiding structure may be formed by e.g. one or more axially extending elongated tracks on the inner surface of the housing, or equivalent structure. Such equivalent structure may, for example, be an axially elongated groove in the housing, or an axially extending protrusion in the housing. The guiding structure simplifies the insertion of the drive assembly into the housing and ensures that the drive assembly is inserted correctly. In this exemplified embodiment, the drive assembly may preferably comprise means compatible with the drive assembly guiding structure, such as e.g. a notch or protrusion sliding along the track during insertion, etc.

According to an exemplified embodiment, the drive assembly may comprise a biasing member connected to the plunger rod. Preferably, and according to an exemplified embodiment, the biasing member may be arranged to generate the activating force on the plunger rod. Hereby, the biasing member is forcing the plunger rod to expel the medicament to the dose delivery site. Preferably, and according to an exemplified embodiment, the biasing member may be a spring. The spring may preferably be a coil spring generating the activating force in the axial direction of the medicament delivery device.

According to a second aspect, there is provided a method of assembling a subassembly of a medicament delivery device, wherein the subassembly comprises a housing configured to contain a medicament container structure from which medicament is expelled during use of the medicament delivery device, the housing having a proximal end and a sealed distal end, and a drive assembly configured to generate an activating force on the medicament container structure for expelling the medicament when the medicament container structure is arranged in the medicament delivery device, the method comprising the steps of inserting the drive assembly into the housing through an opening of the housing at the proximal end, and moving the drive assembly in a distal direction within the housing towards the sealed distal end of the housing.

Hereby, a simple assembly method is provided where only the proximal end of the housing is open for receiving the drive assembly to form the subassembly. The drive assembly is preferably moved in the distal direction until it is positioned in the vicinity of the sealed distal end of the housing. According to a non-limiting exemplified embodiment, the drive assembly may be moved in the distal direction until a distal end of the drive assembly is arranged in an abutment with an inner surface of the sealed distal portion.

Further effects and features of the second aspect are largely analogous to those described above in relation to the first aspect.

According to a third aspect, there is provided a method of assembling a medicament delivery device, the medicament delivery device comprising a subassembly comprising a housing having a proximal end and a distal end, wherein the proximal end of the housing comprises an opening and the distal end comprises a sealable portion, the subassembly further comprising a drive assembly, and a medicament container structure comprising a medicament, wherein the method comprises the steps of assembling a subassembly as described above in relation to the second aspect, and inserting the medicament container structure into the housing through the opening of the housing at the proximal end.

Hereby, an assembly comprising the subassembly described by the first and second aspects, as well as a medicament container structure is provided, where each one of the subassembly, and the medicament container structure is assembled through the proximal end of the housing.

According to an exemplified embodiment, the medicament delivery device may further comprise an expulsion member, wherein the step of inserting the medicament container structure into the housing is preceded by the step of inserting the medicament container structure into the expulsion member by moving the medicament container structure in a proximal direction relative to the expulsion member, thereby forming a medicament expulsion arrangement, wherein the medicament container structure is inserted into the housing by inserting the medicament expulsion arrangement into the housing through the opening of the housing at the proximal end.

An intermediate assembly step is hereby provided in which the medicament container structure is inserted into the expulsion member before the medicament container structure and the expulsion member, i.e. the medicament expulsion arrangement is inserted into the housing at the proximal end of the housing.

According to an exemplified embodiment, the method may further comprise the step of moving the medicament expulsion arrangement in a distal direction within the housing towards the sealed distal end of the housing. The medicament expulsion arrangement is preferably moved in the distal direction until a distal end of the medicament container structure is in the vicinity of a plunger rod of the drive assembly, such that a gap is present between the distal end of the medicament container structure and the plunger rod. According to an exemplified embodiment, the medicament expulsion arrangement may alternatively be moved in the distal direction until a distal end of the medicament container structure abuts a plunger rod of the drive assembly.

Further effects and features of the third aspect are largely analogous to those described above in relation to the first and second aspects.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1A is a perspective view of a medicament delivery device in an assembled configuration according to an exemplified embodiment;

FIG. 1B is an exploded view of the medicament delivery device in FIG. 1A;

FIG. 2 is an exploded view of components forming part of a medicament delivery device according to another exemplified embodiment;

FIG. 3 is an exploded view of a drive assembly according to an exemplified embodiment;

FIG. 4A is a schematic illustration of a method of assembling a subassembly of a medicament delivery device according to an exemplified embodiment;

FIG. 4B is a flow chart of the method depicted in FIG. 4A;

FIG. 5A is a schematic illustration of a method of assembling a medicament delivery device according to an exemplified embodiment; and

FIG. 5B is a flow chart of the method depicted in FIG. 5A.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

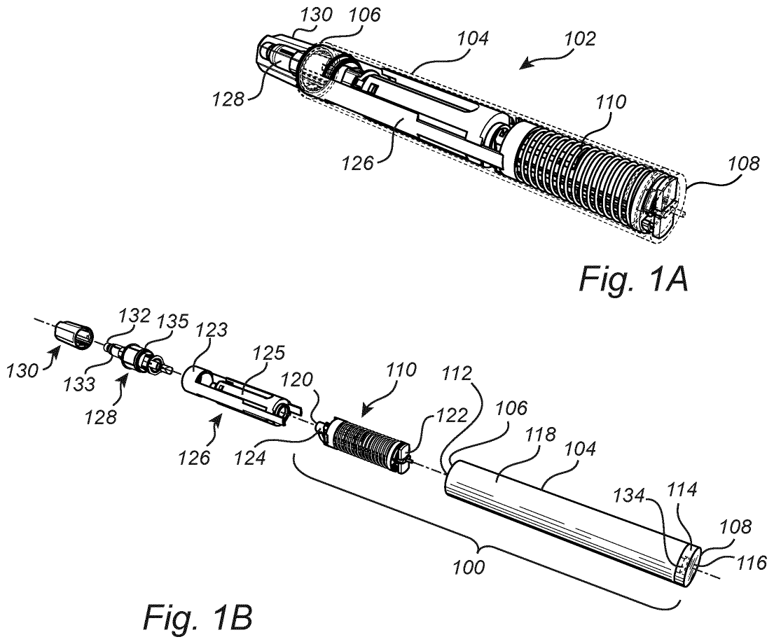

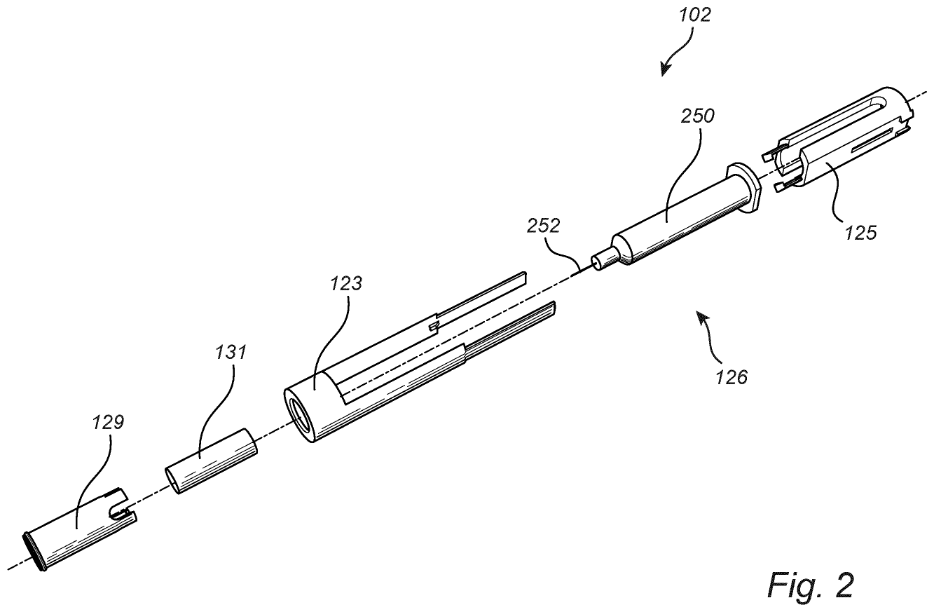

Reference is made to FIGS. 1A and 1B, which illustrate an exemplified embodiment of a medicament delivery device 102 configured to expel medicament from a medicament container structure (shown in detail in FIG. 2). In detail, FIG. 1A illustrates the medicament delivery device 102 in an assembled configuration and FIG. 1B illustrates the medicament delivery device 102 in an exploded view.

As can be seen, the medicament delivery device 102 comprises a housing 104 having a proximal end 106 and a distal end 108. The proximal end 106 of the housing 104 is the end portion of the housing 104 arranged closest to the dose delivery site during use of the medicament delivery device 102 compared to the distal end 108 of the housing 104. The distal end 108 of the housing 104 is thus the end portion of the housing 104 arranged furthest away from the dose delivery site during use of the medicament delivery device 102, as seen in an axial direction of the housing 104.

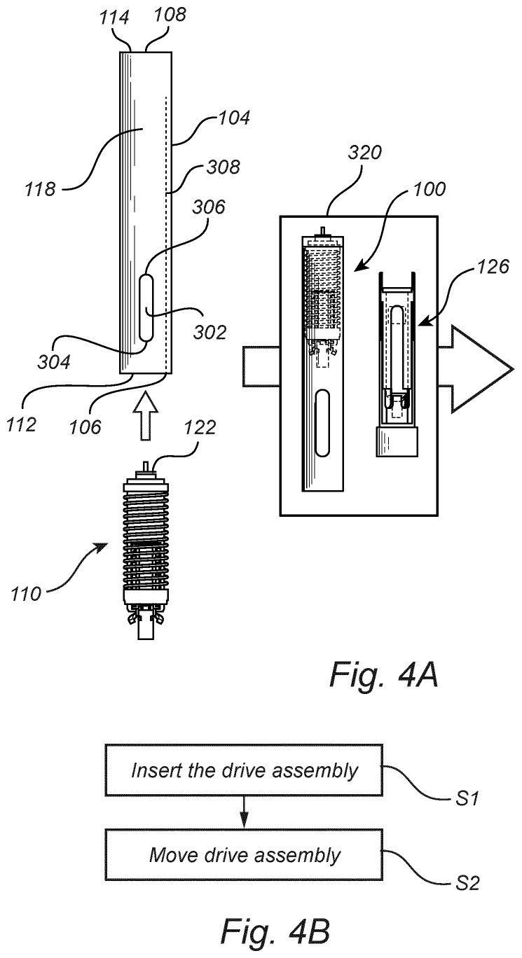

As can be gleaned from FIG. 1B, the housing 104 comprises an opening 112 at the proximal end 106, while the distal end 108 of the housing 104 comprises a sealable portion 114. According to the embodiment depicted in FIGS. 1A and 1B, the sealable portion 114 is formed by a removable cap 116. The removable cap 116 can be provided with external threads (not shown) meshing with internal threads of the housing 104, thereby forming a screw connection between the removable cap 116 and the housing 104. Other alternatives are of course also conceivable, such as e.g. a snap fit connection between the removable cap 116 and the housing 104, or a press-fit connection. As is briefly described below and illustrated in FIG. 4A, the sealable portion 114 may alternatively be integrally formed with an envelope surface 118 of the housing 104. As can be seen in FIG. 1B, the envelope surface 118 extends axially from the proximal end 106 to the distal end 108 of the housing 104.

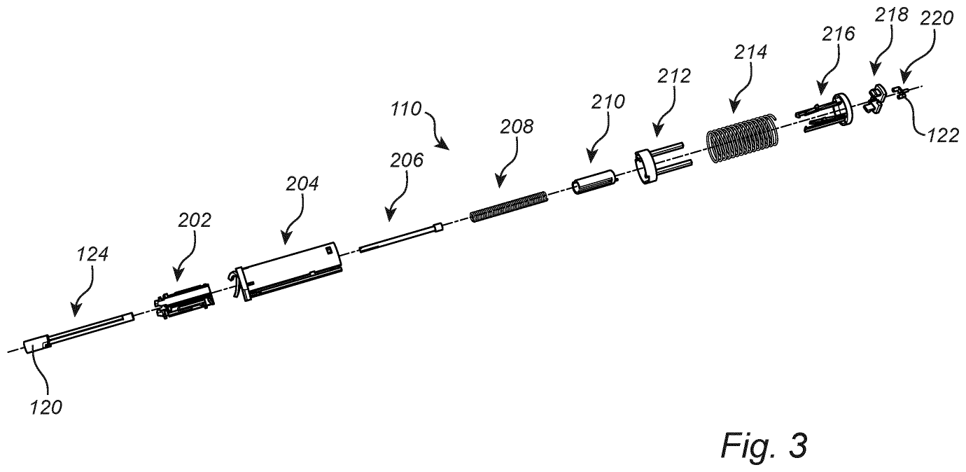

The medicament delivery device 102 further comprises a drive assembly 110. The drive assembly 110 has a proximal end 120 and a distal end 122. The drive assembly 110 and the housing 102 are hereby forming a subassembly 100 of the medicament delivery device 102. The drive assembly 110, which will be described in further detail below with reference to FIG. 3, comprises a plunger rod 124 at the proximal end 120 of the drive assembly 110. During use of the medicament delivery device 102, the plunger rod 124 is configured to apply an activating force on the medicament container structure for expelling the medicament.

Moreover, the medicament delivery device 102 comprises an expulsion member 126, a cartridge 128 and a cartridge cap 130. The cartridge 128 should in the following be understood as a portion of the medicament delivery device 102 that comprises a needle (not shown), which will be further described below. The cartridge 128 may preferable be formed by two or more components connected to each other forming the cartridge 128. In particular, the cartridge 128 comprises a cartridge proximal portion 133 and a cartridge distal portion 135, and the needle is arranged within the cartridge distal portion 135. The cartridge proximal portion 133 is removably connectable to the cartridge distal portion 135. Also, the cartridge 128 preferably comprises a needle hub (not shown) arranged in the cartridge proximal portion 135. The needle hub is arranged to hold the needle. Further, the needle hub is movable relative to the cartridge distal portion 135 and movable for penetrating the medicament container structure (shown in detail in FIG. 2) during use of the medicament delivery device 102. The cartridge cap 130 is a cover that is arranged at a cartridge proximal end 132 of the cartridge 128. Thus, the cartridge cap 130 prevents exposure of the needle when attached to the cartridge 128. Further, the cartridge cap 130 is connected to the cartridge proximal portion 133. In one example, by rotation of the cartridge cap 130 relative to the cartridge distal portion 135 around the needle, the cartridge cap 130 as well as the cartridge proximal portion 133 is removed from the cartridge distal portion 135. Hereby, the needle is exposed for usage of the medicament delivery device, i.e. for penetrating the needle at e.g. a dose delivery site. The expulsion member 126 comprises a needle cover 123 and a container holder 125. The expulsion member 126, and in particular the container holder 125 is configured to house the medicament container structure, and the cartridge 128 is arranged to be connected to the expulsion member 126. As indicated above, the cartridge 128 comprises a needle (not shown) at the cartridge proximal end 132 and the cartridge cap 130 is removably connected to the cartridge 128 at the cartridge proximal end 132. There are thus two types of medicament container structures. In the first exemplified embodiment depicted in FIGS. 1A and 1B, the needle is arranged in the cartridge 128 and arranged to penetrate the medicament container structure before usage of the medicament delivery device 102. Thus, in the first embodiment depicted in FIGS. 1A and 1B, the needle does not form part of the medicament container structure. In a second exemplified embodiment which is described in further detail below, the needle is instead forming part of the medicament container structure.

When the medicament delivery device 102 is assembled as depicted in FIG. 1A, the drive assembly 110 and the expulsion member 126 are arranged within the housing 104. The cartridge 128 is at least partly arranged within the housing 104, while the cartridge cap 130 is arranged outside the housing 104. In detail, and as seen in a direction from the distal end 108 to the proximal end 106, the housing 104 accommodates the drive assembly 110, the expulsion member 126 and the cartridge 128. Furthermore, and as can be seen in FIG. 1B, the sealable portion 114 may comprise a connector portion 134 at a surface of the sealable portion 114 facing the drive assembly 110. The drive assembly 110 can hereby be attached to the sealable portion 114 at the connector portion 134.

Reference is made to FIG. 2 to describe components of a medicament delivery device 102 according to another exemplified embodiment. While the embodiment described above in relation to FIGS. 1A and 1B relates to a so-called cartridge configuration, the FIG. 2 embodiment relates to a so-called syringe configuration. As described above, a difference between the first exemplified embodiment of FIGS. 1A-1B and the second exemplified embodiment of FIG. 2, is that the needle forms part of the cartridge 128 in the first exemplified embodiment, while the needle forms part of the medicament container structure 250 in the second exemplified embodiment.

The medicament delivery device 102 of the exemplified embodiment depicted in FIG. 2 comprises, as seen from the distal end of the housing (housing not illustrated in FIG. 2), the above described container holder 125, the medicament container structure 250 provided with a medicament, a needle cover 123, a rigid needle shield 131, and a shield remover 129. In a similar vein as described above in relation to the cartridge configuration, the needle cover 123 and the container holder 125 are forming parts of an expulsion member 126. In the second exemplified embodiment depicted in FIG. 2, and as indicated above, the medicament container structure 250 comprises a needle 252 at a proximal end of the medicament container structure 250. The needle 252 is thus arranged on the medicament container structure 250 before usage.

The medicament container structure 250 thus comprises the needle 252. The needle 252 is, before being used, arranged in the rigid needle shield 131. The rigid needle shield 131 is an axially extending portion arranged to embed the needle 252 and protect the needle 252 from external damage. The rigid needle shield 131 is in turn housed within the shield remover 129. Further, the medicament container structure 250 is arranged within the container holder 125, and the needle cover 123 is arranged outside the container holder 125 as seen in a radial direction of the medicament delivery device 102. Although not depicted in FIG. 2, the medicament container structure 250, the container holder 125, and the needle cover 123 are all, in an assembled configuration, arranged within a housing 104 similar to the one depicted in FIGS. 1A and 1B. Further, the arrangement in FIG. 2 also preferably comprises a needle cap (not shown) housing the rigid needle shield 131 before use. The needle cap is preferably also connected to the needle cover at a proximal end thereof and removed before usage of the medicament delivery device 102.

Before the medicament delivery device 102 is used, the needle cap (not shown) is removed. When removing the needle cover, the shield remover 129 will also be removed, forcing the rigid needle shield 131 to be pulled away from the needle 252, thereby exposing the needle 252. An exemplified embodiment of how the medicament delivery device 102 is being prepared for usage is given below with reference to FIG. 3.

A difference between the second embodiment depicted in FIG. 2 and the first embodiment depicted in FIGS. 1A and 1B is that the needle 252 is in the second exemplified embodiment arranged on the medicament container structure 250. In the first exemplified embodiment depicted in FIGS. 1A and 1B, the needle is instead forming part of the cartridge 128. The first embodiment in FIGS. 1A and 1B thus comprises a medicament container structure with no needle attached to it. Instead, the needle in the cartridge 128 penetrates the medicament container structure during use of the medicament delivery device 102.

To describe the drive assembly 110 in further detail, reference is now made to FIG. 3 which is an exploded view of the drive assembly 110 according to an exemplified embodiment. It should be readily understood that the features of the drive assembly 110 in FIG. 3 is merely for illustrating and describing a conceivable embodiment of the drive assembly 110. The drive assembly 110 may be arranged in a number of different configurations, with either further or less components compared to the FIG. 3 embodiment.

According to the FIG. 3 embodiment of the drive assembly 110, the drive assembly 110 comprises the above described plunger rod 124 at the proximal end 120 of the drive assembly 110. The drive assembly 110 further comprises a slider element 202, a drive assembly frame structure 204, a plunger rod guidance member 206 and a biasing member 208. The biasing member 208 is preferably, and as depicted in FIG. 3, a spring arranged in the form of a coil spring. The drive assembly 110 also comprises an end click member 210, a sleeve 212, an activation spring 214, a start click member 216, a frame cover 218 and a splint 220.

The following will briefly describe the functionality of the drive assembly 110. Thus, a detailed description of the interaction between all components of the drive assembly will not be given. Since the drive assembly can be arranged in a number of different configurations, only the overall functionality will hereby be provided. When the medicament delivery device 102 is arranged from a starting position, i.e. before the cartridge cap 130 is removed from the cartridge 128, to an injection position, i.e. when the cartridge cap 130 is removed from the cartridge 128, the sleeve 212 is moved in the distal direction along the drive assembly frame structure 204, which drive assembly frame structure 204 is kept stationary relative to the sleeve 212. According to an example, a proximal end of the sleeve 212 is, in an assembled configuration of the medicament delivery device 102 as depicted in e.g. FIG. 1A, arranged in abutment with a distal end of the needle cover 123. The sleeve 212 is thus moved in the distal direction when a user exerts an axial force axially moving the needle cover 123 relative to the drive assembly frame structure 204. The activation spring 214 is arranged in abutment with the sleeve 212 and the start click member 216. The sleeve 212 interacts with the activation spring and by moving the sleeve 212 in the distal direction, the activation spring 214 will compress and slightly move also the start click member 216 distally. The activation spring 214 is compressed until the start click member 216 is arranged at an end position in the vicinity of the distal end 122 of the drive assembly 110. The medicament delivery device 102 is now ready for injecting medicament into the dose delivery site. Thereafter, when the medicament is to be expelled from the medicament container structure, the drive assembly 110 is activated whereby the biasing member 208 pushes the plunger rod 124 onto the medicament container structure generating an activating force on the medicament container structure. The activating force hereby forces the medicament out from the medicament container structure and out through the needle.

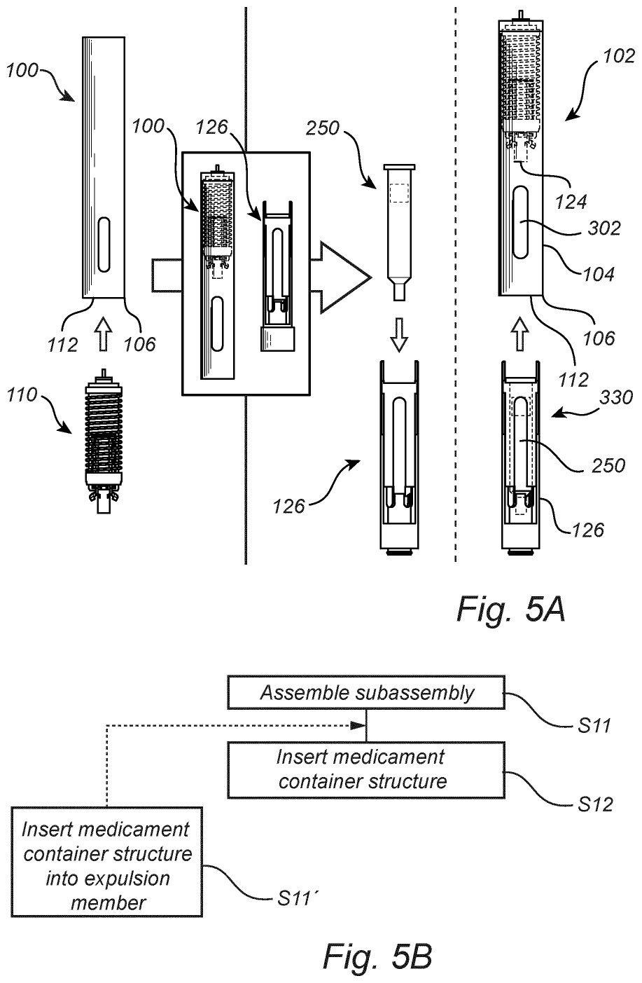

The following will now describe the method of assembling the subassembly 100 described above in relation to FIGS. 1A-1B and FIG. 2. Reference is therefore made to FIGS. 4A and 4B, in which FIG. 4A is a schematic illustration of the method of assembling the subassembly 100 of the medicament delivery device 102 according to an exemplified embodiment, and FIG. 4B is a flow chart of the exemplified method. The description in relation to FIGS. 4A and 4B, as well as in relation to FIGS. 5A and 5B are given in relation to the syringe configuration described above in relation to FIG. 2. It should however be readily understood that the following disclosure is equally applicable for the cartridge configuration described above in relation to FIGS. 1A and 1B.

As can be seen in the FIG. 4A embodiment, the housing 104 here further comprises an elongated inspection portion 302. The elongated inspection portion 302 is arranged in the vicinity of the proximal end 106 of the housing 104. In further detail, a proximal end 304 of the inspection portion 302 is arranged closer to the proximal end 106 of the housing compared to the distance to the distal end 108 of the housing 104. As also depicted in FIG. 4A, and as indicated above, the sealable portion 114 is integrally formed with the envelope surface 118 of the housing 104. Hence, the sealable portion 114 and the envelope surface 118 are formed in one piece. Still further, the FIG. 4A embodiment also comprises a drive assembly guiding structure 308 on the inner surface of the housing 104. The drive assembly guiding structure 308 is in the exemplified embodiment of FIG. 4A arranged as an elongated trace extending between the proximal 106 and distal 108 ends within the housing 104.

During assembly of the subassembly 100, the drive assembly 110 is inserted S1 into the opening 112 arranged at the proximal end 106 of the housing 104. In particular, the drive assembly 110 is inserted into the opening 112 by means of inserting the distal end 122 of the drive assembly 110 into the opening 112. The drive assembly 110 is thereafter moved S2 within the housing 104 in the distal direction towards the distal end 108 of the housing 104. Hereby, when the subassembly 100 is assembled, the drive assembly is arranged distally from the distal end 306 of the inspection portion 302 as can be seen in the assembled configuration depicted within the square box 320. According to a non-limiting example, the drive assembly can be moved all the way within the housing 104 until the distal end 122 of the drive assembly abuts the inner surface of the sealable portion 114.

The subassembly 100 can hereby be delivered, together with the expulsion member 126, to e.g. another production site or to an end user of the medicament deliver device 102. Thus, the assembled subassembly 100 is preferably not connected to the expulsion member 126 at this stage.

Reference is made to FIGS. 5A and 5B for describing a method of assembling the medicament delivery device 102. More detailed, FIG. 5A is a schematic illustration of a method of assembling the medicament delivery device 102 according to an exemplified embodiment, while FIG. 5B is a flow chart of the method depicted in FIG. 5A.

As an initial step, the subassembly is assembled S11 in the manner described above in relation to FIGS. 4A and 4B. Hence, the drive assembly 110 is inserted through the opening 112 at the proximal end 106 of the housing to form the subassembly 100. The medicament container structure 250 is thereafter inserted S12 into the housing 104 through the opening 112 of the proximal end 106 of the housing 104.

Preferably, and as depicted in FIG. 5A, before the medicament container structure 250 is inserted into the housing 104, the medicament container structure 250 is inserted S11′ into the expulsion member 126. In particular, the medicament container structure 250 is inserted into the expulsion member 126 by moving the medicament container structure 250 in a proximal direction relative to the expulsion member 126. The medicament container structure 250 and the expulsion member 126 hereby forms a medicament expulsion arrangement 330. The step S12 of inserting the medicament container structure 250 into the housing 104 at the opening 112 of the proximal end 106 is hereby executed by moving the medicament expulsion arrangement 330 into the housing 104 through the opening 112 at the proximal end 106 of the housing 104.

The medicament expulsion arrangement 330, and thus the medicament container structure 250 is thereafter moved within the housing 104 in the distal direction until arriving at a final assembled position. Preferably, the assembled position is reached when the medicament container structure abuts the plunger rod 124 of the drive assembly 110, or is arranged in the vicinity of the plunger rod 124 of the drive assembly 110. According to another example, the arrangement may comprise the above described cartridge cap 130, whereby the final assembled position is reached when the cartridge cap 130 abuts on the proximal end 106 of the housing 104.

When the medicament delivery device 102 is finally assembled, the medicament container structure 250 will be arranged at the inspection portion 302 of the housing 104, for enabling inspection by the user of the medicament delivery device

According to some variations, the drive assembly could, instead of the above described automatic spring-loaded structure, for example be motor driven or manually driven. Moreover, it is envisaged that the medicament container structure 250 could be inserted into the housing 104 without being arranged within the expulsion member 126. For example, in the case of using the cartridge configuration depicted in FIGS. 1A and 1B, the cartridge 128 can, together with the medicament container structure 250, be inserted into the housing 104 directly without the use of the expulsion member 126. In such a case, the cartridge can, for example, be supported by either an inner frame of the housing or supported by a part of the cartridge cap 130.

The inventive concept has mainly been described above with reference to a few examples. However, as is readily appreciated by a person skilled in the art, other embodiments than the ones disclosed above are equally possible within the scope of the inventive concept, as defined by the appended claims.

1-15. (canceled)

16. A subassembly of a medicament delivery device, the subassembly comprising:
a housing configured to contain a medicament container structure from which medicament is expelled during use of the medicament delivery device, the housing having a proximal end and a distal end; and
a drive assembly comprising a plunger rod at a proximal end of the drive assembly, the plunger rod being configured to, upon activation, apply an activating force on the medicament container structure for expelling the medicament from the medicament delivery device,
wherein the proximal end of the housing comprises an opening, and the distal end comprises a sealable portion, wherein the drive assembly is insertable into the housing at the proximal end to form the subassembly.

17. The subassembly according to claim 16, wherein the housing comprises an envelope surface extending axially from the distal end to the proximal end, the sealable portion being integrally formed with the envelope surface.

18. The subassembly according to claim 16, wherein the sealable portion is formed by a removable cap.

19. The subassembly according to claim 16, wherein a distal end of the drive assembly is arranged at the distal end of the housing when the drive assembly is inserted into the housing.

20. The subassembly according to claim 16, wherein the housing is a cylindrical housing forming a hollow housing structure.

21. The subassembly according to claim 16, wherein the housing comprises an inspection portion.

22. The subassembly according to claim 21, wherein the inspection portion is an elongated inspection portion extending along an axial direction of the housing, wherein a proximal end of the inspection portion is arranged closer to the proximal end of the housing compared to the distal end of the housing.

23. The subassembly according to claim 16, wherein the sealable portion comprises a connector portion at a surface facing the drive assembly.

24. The subassembly according to claim 16, wherein the housing comprises a drive assembly guiding structure, the drive assembly guiding structure extending in an axial direction on an inner surface of the housing.

25. The subassembly according to claim 16, wherein the drive assembly comprises a biasing member connected to the plunger rod.

26. The subassembly according to claim 25, wherein the biasing member is arranged to generate the activating force on the plunger rod.

27. A method of assembling a subassembly of a medicament delivery device, wherein the subassembly comprises a housing configured to contain a medicament container structure from which medicament is expelled during use of the medicament delivery device, the housing having a proximal end and a sealed distal end, and a drive assembly configured to generate an activating force on the medicament container structure for expelling the medicament when the medicament container structure is arranged in the medicament delivery device, the method comprising the steps of:
inserting the drive assembly into the housing through an opening of the housing at the proximal end; and
moving the drive assembly in a distal direction within the housing towards the sealed distal end of the housing.

28. A method of assembling a medicament delivery device, the medicament delivery device comprising:
a subassembly comprising a housing having a proximal end and a distal end, wherein the proximal end of the housing comprises an opening and the distal end comprises a sealable portion, the subassembly further comprising a drive assembly, and a medicament container structure comprising a medicament, wherein the method comprises the steps of:
assembling the subassembly according to claim 27; and
inserting the medicament container structure into the housing through the opening of the housing at the proximal end.

29. The method according to claim 28, wherein the medicament delivery device further comprises an expulsion member, wherein the step of inserting the medicament container structure into the housing is preceded by the step of:
inserting the medicament container structure into the expulsion member by moving the medicament container structure in a proximal direction relative to the expulsion member, thereby forming a medicament expulsion arrangement,
wherein the medicament container structure is inserted into the housing by inserting the medicament expulsion arrangement into the housing through the opening of the housing at the proximal end.

30. The method according to claim 29, further comprising the step of:
moving the medicament expulsion arrangement in a distal direction within the housing towards the sealed distal end of the housing.
